(19) United States
(12) Patent Application Publication
(10) Pub. No.: US 2024/0092863 A1
(43) Pub. Date: Mar. 21, 2024

(54) ANTIMICROBIAL TARGET

(71) Applicant: UNIVERSITY OF SHEFFIELD, Sheftield (GB)

(72) Inventors: Peter Nigel Monk, Sheffield (GB); Rahaf Issa, Manchester (GB); Barbara Ciani, Sheffield (GB)

(21) Appl. No.: 17/909,318

(22) PCT Filed: Mar. 2, 2021
(86) PCT No.: PCT/EP2021/055109

§ 371 Date: Sep. 2, 2022

(30) Foreign Application Priority Data
Mar. 3, 2020 (GB) 2003062.3

(51) Int. Cl. C07K 14/705; A61P 31/04

(52) U.S. Cl. CPC C07K 14/70596; A61P 31/04

(57) ABSTRACT

The present invention relates to a peptide for reducing pathogen adhesion. Specifically, a peptide comprising an amino acid sequence having at least 75% sequence identity to SEQ ID NO: 1. The peptide also comprises at least one staple between two or more residues equivalent to residues 3, 7, 10 or 14 of SEQ ID NO: 1.

### CROSS-REFERENCE TO RELATED APPLICATION(S)

This application is a national phase entry of PCT/EP2021/055109, filed on Mar. 2, 2021, which claims priority to GB 2003062.3, filed on Mar. 3, 2020, the entire contents of each of which are fully incorporated herein by reference.

### REFERENCE TO SEQUENCE LISTING

This application was filed with a Sequence Listing in text format in accordance with 37 C.F.R. § 1.821. The Sequence Listing text file submitted in the USPTO Patent Center, “217567-0004-US01-Sequence-Listing.txt,” was created on Apr. 24, 2023, contains 18 sequences, has a file size of 5723 bytes, and is incorporated by reference in its entirety into the specification.

### FIELD OF INVENTION

The present invention relates to a peptide, or a pharmaceutical composition comprising said peptide, having at least one staple in a defined location in the peptide, which shows increased efficacy and/or stability compared to the wild type protein. Aspects of the invention further relate to use of such a peptide, or pharmaceutical composition comprising said peptide, to treat a wound or a bacterial infection, in particular in the skin or the cornea.

### BACKGROUND TO THE INVENTION

Antimicrobial resistance (AMR) is a serious and growing problem. Many pathogens, such as Staphylococcus aureus (S. aureus) are common causative pathogens in wound infections, but can progress from symptoms including dryness, pruritus, and pain, to more severe effects such as cellulitis, folliculitis, furuncles and impetigo, and potentially fatal systemic infection [1].

Like most bacterial skin pathogens, S. aureus has to gain access to target tissues via a break in the stratum corneum and then attach to underlying cells to cause an infection. S. aureus, as also many pathogens, has a range of adhesins that allow it to adhere tightly to molecules associated with the host cell surface. Such host receptor (or ‘adhesion target’) molecules include the extracellular matrix protein fibronectin, scavenger receptors such as CD36 and surface-expressed chaperone Hsc70. Similarly, pathogens must attach and transverse other epithelial layers, such as those at the eye, lung and gut.

Previously, there have been attempts to prevent this adhesion in order to reduce pathogen binding and therefore colonisation and/or infection, such as heparin binding peptides which block heparan sulphates on the host cell surfaces as in U.S. Pat. No. 7,259,140B2, and MAM7 peptide which binds and blocks fibronectin and phosphatidic acid to inhibit Gram negative pathogen adhesion as in EP2693887. A summary of such attempts can be found in a review article by Asadi et al 2019 (Infection; 47:13-23), which notes that much work is need in order to understand the mechanism of adhesion and how this may be targeted. However, many of these previous attempts have generated peptides that have issues that have prevented their commercial exploitation including prohibitive cost, immunogenicity, specificity, and efficacy.

Many host adhesion target molecules are known to interact with a family of eukaryotic membrane proteins known as tetraspanins, which act as molecular facilitators [2]. Tetraspanins are membrane proteins characterized by 4 transmembrane domains, containing charged residues, 1 intracellular loop, 2 intracellular termini and 2 extracellular loops, the second of which (EC2 domain) makes specific protein-protein interactions. Tetraspanins associate with each other in the membrane via membrane-proximal palmitoylation sites, as well as associating with other cell components such as signalling molecules, structural proteins and G-protein coupled receptors, in order to form tetraspanin-enriched microdomains (TEM).

TEM have been implicated in many cell functions, including cell adherence and fusion, membrane trafficking, endocytosis, leukocyte adherence and motility but can also be exploited by protozoa, viruses and bacteria as gateways for infection [3, 4]. For example, uropathogenic Escherichia coli (E. coli) have been shown to exploit tetraspanins in order to adhere to bladder cells through the E. coli adhesin, FimH, binding directly to tetraspanin TSPAN21 [5]. More commonly, bacterial adhesion requires an indirect interaction with tetraspanins, through receptors embedded in TEM [6].

It has been found that short peptides 810, 8001 and 800 derived from the primary sequence of the tetraspanin CD9 large extracellular domain can reduce the adherence of Staphylococcus aureus binding to human keratinocyte cells [7]. In particular, it was found that the 800 peptide was functionally active in a time and dose-dependent manner, and was also effective in lowering bacterial burden in a 3D model of human skin.

Notwithstanding the above, there are inherent issues with the clinical application of such peptides as illustrated in the prior art, including issues with degradation and lack of activity, as well as clinical storage. In addition, most research in the anti-pathogen field so far has been focused on attacking the pathogen through, for example, antibiotics or phage therapy, rather than affecting the adhesion of the pathogen to the host. Accordingly, there is a need to produce an agent that can reduce infections and pathogen load issues, whilst overcoming AMR and showing good activity and stability.

### SUMMARY OF THE INVENTION

Using rational design to modify the 800 peptide derived from the extracellular domain of CD9 we have generated stapled peptides that have surprisingly improved activity as well as stability, improving the reduction in pathogen adhesion.

Accordingly, in one aspect of the invention there is provided a peptide comprising an amino acid sequence having at least 75% sequence identity to SEQ ID NO: 1, wherein the peptide comprises at least one staple between two or more residues equivalent to positions 3, 7, 10 or 14 (‘Xaa’) of SEQ ID NO: 1.

The skilled person will understand that a staple is formed by a covalent linkage between two amino acid side-chains, forming a stapled peptide. Stapling has been used to enhance the pharmacologic performance of peptides. The skilled person will also understand that stapling is a strategy for constraining short peptides typically in an α-helical formation. Or, in other words, stapling is a strategy for increasing the propensity of a peptide to be in α-helical formation.

Without wishing to be bound by theory, we believe that in some embodiments, the stapled peptide of the invention forms an α-helix in aqueous solution. In one embodiment, the peptide forms an α-helix when in an aqueous solution. An aqueous solution may be any solution comprising water. An aqueous solution may consist of at least 20%, 30%, 40%, 50%, 60%, 70%, 80%, or 90% water, or may consist of 100% water. An aqueous solution that comprises less than 100% water may also comprise another solvent such as an alcohol, including methanol, ethanol, trifluoroethanol or hexaisopropanol. In some embodiments, the aqueous solution is 5% alcohol and 95% water, 10% alcohol and 90% water, 25% alcohol and 75% water, 50% alcohol and 50% water, 75% alcohol and 25% water, or 100% alcohol; preferably wherein, the alcohol is methanol, ethanol, trifluoroethanol or hexaisopropanol.

The Xaa residues in SEQ ID NO: 1 may independently be any amino acid. Preferably, each Xaa may independently be any hydrophilic polar amino acid, and more preferably each Xaa may independently be any hydrophilic polar amino acid. For example, each Xaa may be independently selected from threonine, proline, glutamine, asparagine, and alanine. More preferably, each Xaa may independently be an alanine. Most preferably the Xaa at position 10 and Xaa at position 14 are alanine, and the Xaa at position 3 and Xaa at position 7 may be either alanine or the Xaa position 3 is proline and Xaa position 7 is threonine.

The peptide of the invention comprises at least one staple between two Xaa residues at any of positions 3, 7, 10 and 14. In preferred embodiments, the staple is between residues 3 and 7, or the staple is between residues 10 and 14; however, the staple may instead be between residues 3 and 10; or 7 and 14; or 3 and 14. In preferred embodiments, both of these Xaa residues are identical; in most preferred embodiments, each Xaa is alanine. It will be clear to the skilled person that the amino acids at these Xaa residues to be stapled may require modification in order to accommodate said staple. Therefore, in some embodiments the Xaa residues may be modified versions of amino acids, wherein the skilled person recognises that the modified versions of these amino acid are so modified to accommodate a staple. For example, the modified amino acid may be an olefin-terminated modified amino acid; conveniently, the modified amino acid may include a carbon chain of 5, 6, 7, 8, 9, or 10 carbons. Accordingly, in some embodiments, the Xaa residue is (S)-2-(4-pentenyl)-alanine, (S)-2-(7-octenyl)-alanine, or (S)-2-(4-pentenyl)-glycine. S entantiomers or R enantiomers may be used. The two Xaa residues that are not stapled may be any amino acid, but preferably will be the wild type amino acid found in the same position of SEQ ID NO: 5. In other words, if position 3 is not stapled then this residue may be proline, if position 7 is not stapled then this residue may be threonine, if position 10 is not stapled then this residue may be alanine, and if position 14 is not stapled then this residue may be alanine.

In one aspect, the peptide consists of no more than 16, 17, 18, 19 or 20 amino acids. In one aspect of the invention, the peptide consists of an amino acid sequence having at least 75% sequence identity to SEQ ID NO:1, wherein the peptide comprises at least one staple between two or more residues equivalent to residues 3, 7, 10 or 14 of SEQ ID NO:1.

In some embodiments, the peptide comprises or consists of an amino acid sequence having at least 80%, 85%, 90%, 95%, or 99% sequence identity to SEQ ID NO: 1.

It will be understood that the peptide falling within this definition has the effect of the invention, or specifically, reducing pathogen adhesion to cells. It will also be understood that a peptide having at least 90%, 95% or 99% sequence identity to SEQ ID NO: 1 will also fall under the scope of the invention.

In a further embodiment, the stability and/or the activity of the peptide is increased compared to a peptide consisting of any one of SEQ ID NO: 5, 6 or 7. It will be understood that a peptide consisting of SEQ ID NO: 5, 6 or 7 relates to the wild type (and unstapled) 800 peptide sequence in human, mouse or pig respectively. In one example, activity and/or stability of the peptide of the invention is increased compared to a control, wherein the control may be a non-stapled version of the peptide or may be a wild-type 800 peptide It will therefore be understood that either stability, or activity, or both stability and activity, may be increased compared to a peptide consisting of SEQ ID NO; 5, 6 or 7. Activity and/or stability may be increased by at least 0.5, 1, 2, 5, 10, 20, 50, 100, 200, 500 or 1000 fold compared to a peptide consisting of SEQ ID NO; 5, 6 or 7, or another control peptide as described above.

In another embodiment, the peptide consists of an amino acid sequence defined by SEQ ID NO: 2 or SEQ ID NO: 3, or consists of an amino acid sequence consisting of SEQ ID NO: 1, SEQ ID NO: 2 or SEQ ID NO: 3 having at least one substitution, deletion or addition. It will be understood that at the term “at least” means that the peptide may comprise more than one such modification; for example, the peptide may comprise one, two, three or four modifications.

In some embodiments, the at least one substitution is a conservative mutation, meaning that an amino acid is replaced with another amino acid with similar biochemical properties (e.g. charge and/or hydrophobicity) so that the protein structure and function is the same. In other embodiments the at least one substitution is a non-conservative mutation, meaning that an amino acid is replaced with another amino acid with dissimilar biochemical properties so that the protein structure and function may be affected. In some embodiments, the at least one substitution results in a retro inverso peptide, meaning that an L-amino acid is replaced with a D-amino acid and the sequence reversed.

In some embodiments, the at least one deletion is deletion of residue 1 of SEQ ID NO: 1 (or equivalent residues in SEQ ID NO: 2 or SEQ ID NO: 3), which the person skilled in the art will understand to be deletion of the cap residue.

In some embodiments, the at least one addition may be an addition of a single amino acid to the N-terminus or C-terminus of the peptide of the invention.

In yet another embodiment, the at least one staple comprises a covalent linkage between side chains of said two or more amino acids, preferably wherein at least one of the two or more amino acids is an amino acid having an unnatural side chain. In preferred embodiments, at least two amino acids are amino acids having an unnatural side chains.

A covalent linkage may be understood to be a bond wherein there is sharing of electron pairs between atoms. The term “unnatural side chain” may be defined as a side chain that is not seen in a proteinogenic amino acid, or in other words, a side chain that is not naturally seen in alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. Alternatively, “unnatural side chain” may be defined as a side chain that is not seen in any natural amino acid occurring in the CD9 protein. In some embodiments, an amino acid having an unnatural side chain is a modified amino acid as explained above, such as (S)-2-(4-pentenyl)-alanine, (S)-2-(7-octenyl)-alanine, or (S)-2-(4-pentenyl)-glycine.

In another aspect of the invention, there is provided a peptide as described above for use in therapy. The terms “treatment” or “therapy” may be used interchangeably and refer to any partial or complete treatment and includes: inhibiting the disease or symptom, i.e. arresting its development; and relieving the disease or symptom, i.e. causing regression of the disease or symptom.

In some embodiments, the peptide is within a kit, or a composition, comprising at least one further drug, preferably an antimicrobial, for use in therapy. In some embodiments, the at least one further antimicrobial is an antibiotic. Antibiotics may include any known to the person skilled in the art, for example, the antibiotic may be a penicillin, a cephalosporin, a macrolide, a fluoroquinolone, a sulfonamide, a tetracycline or a aminoglycoside. The skilled person will be aware of many antibiotics within these classes, however by way of example, a penicillin may be amoxicillin or penicillin G; a cephalosporin may be cephalexin or ceftazidime; a macrolide may be azithromycin or erythromycin; a fluoroquinolone may be ciprofloxacin or levofloxacin; a sulfonamide may be co-trimoxazole or mafenide; tetracycline may be doxycycline or tetracycline; and aminoglycoside may be gentamicin or streptomycin.

In one embodiment, the peptide is for treatment of a wound, preferably wherein the wound is a wound to an epithelial layer. For example, a skin wound or a corneal wound. In one embodiment, the peptide is for use in reducing a bacterial load. It will be clear to the skilled person that there may be examples wherein the peptide is being used as both a treatment for a wound and for reducing a bacterial load.

In another aspect of the invention, there is provided a peptide as described above for prevention and/or treatment of a microbial infection. In some embodiments, the microbial infection to be prevented or treated comprises a bacterial or fungal infection. In preferred embodiments, the microbial infection is a pathogenic infection. Optionally, the microbial infection may comprise species from Staphylococcus, Streptococcus, Candida, Fusarium, Pseudomonas, Tricophyton, or Aspergillus. In some embodiments, the microbial infection comprises a single microbial species; in other embodiments, the microbial infection comprises multiple microbial species. In specific embodiments, the infection comprises Staphylococcus aureus and/or Streptococcus pneumoniae. It will be understood that the microbial infection to be prevented or treated may be located in an area of the body. Accordingly, in some embodiments, the microbial infection to be prevented or treated is located in the lungs or other parts of the respiratory system, the peritoneum, or the skin. In one embodiment, prevention and/or clearance of a biofilm is increased when the peptide is used compared to when the peptide is not used.

In another aspect of the invention, there is provided a pharmaceutical composition comprising the peptide as described above. The term “pharmaceutical composition”, as used herein, means a pharmaceutical preparation suitable for administration to an intended human or animal subject for therapeutic purposes. Optionally, a pharmaceutical composition may further comprise a pharmaceutically acceptable carrier such as an excipient or other component that facilitates processing of the active compounds into preparations suitable for pharmaceutical administration.

In a further aspect of the invention, there is provided a use of the peptide described above in the manufacture of a medicament. The term “medicament” means a composition suitable for treatment or therapy as defined above. Again, this use may further comprise a pharmaceutically acceptable carrier as per the above.

In one aspect of the invention, there is provided a use of the peptide as described above in the manufacture of a medicament for treatment of a wound, preferably a wound to an epithelial layer, optionally a skin wound or a corneal wound.

In another aspect of the invention, there is provided a use of the peptide as described above in the manufacture of a medicament for prevention and/or treatment of a microbial infection. In some embodiments, the microbial infection comprises a bacterial or fungal infection. In preferred embodiments, the microbial infection is a pathogenic infection. Optionally, the microbial infection may comprise species from Staphylococcus, Streptococcus, Candida, Fusarium, Pseudomonas, Tricophyton, or Aspergillus. In some embodiments, the microbial infection comprises a single microbial species; in other embodiments, the microbial infection comprises multiple microbial species. In specific embodiments, the infection comprises Staphylococcus aureus and/or Streptococcus pneumoniae. It will be understood that the microbial infection to be prevented or treated may be located in an area of the body. Accordingly, in some embodiments, the microbial infection to be prevented or treated is located in the lungs or other parts of the respiratory system, the peritoneum, or the skin.

In one embodiment, the prevention and/or clearance of a biofilm is increased compared to when the peptide is not used. In another embodiment, the use further comprises use of at least one antimicrobial drug, preferably an antibiotic. Antibiotics may include any known to the person skilled in the art, as described above.

In another aspect of the invention, there is provided a kit, or a composition, comprising a peptide described above and at least one further antimicrobial drug, preferably an antibiotic.

In one aspect of the invention, there is provided a method of treating a wound using the peptide or the pharmaceutical composition as described above. Optionally, the wound is a skin wound or a corneal wound.

In another aspect of the invention, there is provided a method of treating a microbial infection using the peptide or the pharmaceutical composition as described above. Optionally, as above, the microbial infection comprises species from Staphylococcus, Streptococcus, Candida, Fusarium, Pseudomonas, Tricophyton, or Aspergillus. In some embodiments, the microbial infection comprises a single microbial species; in other embodiments, the microbial infection comprises multiple microbial species. In specific embodiments, the infection comprises Staphylococcus aureus and/or Streptococcus pneumoniae. It will be understood that the microbial infection to be prevented or treated may be located in an area of the body. Accordingly, in some embodiments, the microbial infection to be prevented or treated is located in the lungs or other parts of the respiratory system, the peritoneum, or the skin.

In one embodiment, the prevention and/or clearance of a biofilm is increased compared to when the peptide or the pharmaceutical composition is not used.

In another embodiment, the method further comprises use of at least one antimicrobial drug, preferably an antibiotic. As above, antibiotics may include any known to the person skilled in the art, for example any detailed in the list above.

In one embodiment, in a use or method described above the peptide or pharmaceutical composition is administered topically, through inhalation, or by lavage fluid. It will be understood that the administration route will relate to the desired target of the composition, for example, the skin or the cornea by topical administration, the respiratory system though inhalation, or the peritoneal cavity by lavage fluid. It will be further understood that the administration route will further relate to the desired treatment, for example, treatment of epithelial wounds by topical administration, prevention or treatment of microbial infection in the respiratory system through inhalation, and prevention or treatment of microbial infection in the peritoneum by lavage fluid. It will also be understood that the peptide can be formulated to be suitable for any of the above administration routes.

### BRIEF DESCRIPTION OF THE FIGURES

FIG. 1 shows the structure-function relationships in peptide 800.

(A) Representation of the 800 cap peptide within the extracellular domain of the EC2 domain of CD9 and a helical wheel representation of peptide 800-Cap. A38, A34, T31 and P27 are located together on one face of a presumed alpha-helical peptide. Residues 34, 27, 38, 31, 28 and 40 are hydrophilic polar amino acids; residues 35, 39, 32 and 37 are hydrophobic non-polar amino acids; and the remaining residues 25, 36, 29, 33, 36 and 30 are charged amino acids. (B) Testing of peptides at 200 nM on S. aureus adhesion to HaCaT human skin keratinocyte cells. Data are means+/−SEM from three separate experiments conducted in triplicate. Significance of difference from untreated control: *p<0.1; **p<0.01; ***p<0.001; ****p<0.0001, one way ANOVA with Dunnett's post test. (C) Dose-response curves for peptides 800, 800-Cap and stapled peptide 800ii. Data are the means+/−SEM of 3-5 separate experiments performed in duplicate.

FIG. 2 shows a comparison of CD9 EC2 peptide efficacy and potency on bacterial adhesion to human corneal epithelial cells.

P. aeruginosa PA01, S. aureus MRSA6 and S. pneumoniae D39 adherence to HCE-2 human corneal epithelial cells was measured in the presence of different concentrations of 800-CAP, 800i and 800ii. (A) Maximum inhibition of adhesion (Imax) and (B) the concentration of peptide required to produce 50% of the maximum inhibitory response (IC50) are shown for each pathogen. Data are the means+/−SEM from at least 5 separate experiments performed in duplicate. Significance of differences: **p<0.01; ***p<0.001; ****p<0.0001 one way ANOVA with Tukey's post test.

FIG. 3 shows the toxicity of CD9 EC2 peptides.

Potential toxicity of CD9 EC2 peptides was measured on HCE-2 human corneal epithelial cells using (A) HCE-2 cell adhesion, assessed by crystal violet staining after plating suspended cells in the presence of 200 nM and 1000 nM peptides. The data are the means+/−SEM of 5 separate experiments conducted in triplicate; (B) Cytotoxicity, assessed by LDH release (left panel) and metabolic activity, assessed by MTT reduction (right panel) after 24 hours treatment with different concentrations of the indicated peptide. Data are the means from a single experiment conducted in triplicate.

FIG. 4 shows the stability of CD9 EC2 peptides to proteases.

(A) Peptides at 100 ug/ml were incubated in 100 mM ammonium bicarbonate+2 mM calcium chloride were digested using trypsin (2 mg/ml), chymotrypsin (2 mg/ml) or human serum (25% (v/v)) for 1 and 18 h. Digestion was performed at 37° C. for trypsin and human serum, and 25° C. for chymotrypsin. Reaction was stopped with 2.5% formic acid for trypsin and chymotrypsin. Human serum proteins were precipitated with acetonitrile. Supernatants were analysed on LC-MS. (B) Peptides at 200 nM were incubated with mid-log phase S. aureus SH1000 or P. aeruginosa PA01 cultures for 4 hrs before the bacteria were removed by centrifugation and the peptides used for an infection assay using S. aureus SH1000 on HaCaT keratinocytes. The data are the means from 6 or 12 separate experiments performed in duplicate. Significance of difference from 800-CAP for each bacterium was assessed by two way ANOVA. **p<0.01; ***p<0.0005; ****p<0.0001

FIG. 5 shows the effect of CD9 EC2 peptides on bacterial adherence to professional phagocytes.

Human monocyte-derived macrophages from a single donor were incubated with S. aureus SH1000 in the presence or absence of 200 nM peptide 800-Cap and its SCR control for 1 hour. Some samples were then treated with gentamicin to kill extracellular bacteria and left to incubate for a further 4 or 18 hours. (A) Shows adherent bacteria at 1 hour; (B) Intracellular bacteria 4 hours after gentamicin treatment; (C) Intracellular bacteria after 18 hours. Viable bacteria were counted as CFU and normalised to the untreated (infection only) control. Data show the means+/−SEM of six separate experiments conducted in duplicate.

FIG. 6 shows the activity of CD9 EC2 stapled peptide 800i on human skin infections.

(A) A 3D model of human skin was produced using primary human keratinocytes and fibroblasts. After wounding, 100 ul of peptide 800i was added at 200 nM. After 1 hour, S. aureus SH1000 was used to infect the skin for a further 24 hours. Skin was then minced, dissolved in saponin and CFU counted. (B) Shows the CFU per mg of skin in untreated and peptide treated samples. The data were normalised to the infection only control, with means+/−SD of 4 separate experiments performed in duplicate. **p<0.01, one sample t-test vs infection only control.

FIG. 7 shows the activity of CD9 EC2 stapled peptide 800ii on human corneal infections.

Expired human corneas were obtained from the transplant programme at LV Prasad Eye Institute, Hyderabad, India. Corneas were treated with 200 ul 400 nM peptide 800ii and then infected with 200 ul PBS containing 5×10⁶ P. aeruginosa clinical isolate LVP3 for 24 hours. (A) Infected corneas show increasing opacity, which is lower in 800ii-treated corneas. (B) Corneas were minced and dissolved in saponin and CFU counted. Data are the means of 3-4 separate experiments with 2-3 corneas per experiment. **p<0.01, one sample t test vs untreated control.

FIG. 8 shows the activity of CD9 EC2 stapled peptide 800ii on a mouse model of microbial keratitis.

Peptide 800ii can significantly decrease the infection of mouse eyes by P. aeruginosa clinical isolate LVP3 in a mouse model of microbial keratitis. Data was analysed using a ROUT test to eliminate outliers, using Q=10%, and then significance of difference from the saline-treated controls calculated using ANOVA with Dunnett's post test. (A) A single application of either 500 nM or 1000 nM 800ii caused a significant reduction in the bacterial load after 24 hrs. *p<0.05. (B). In addition, 800ii enhances wound healing in mouse corneas. Corneas were cut with a scalpel and allowed to heal for 24 hrs after treatment with saline (control) or 1000 nM peptide 800ii. The wound score was assessed using fluorescein permeability (0=no permeability; 1=permeability). Significance of difference from 1 calculated by one-sample t test, *p<0.05.

FIG. 9 shows the activity of CD9 EC2 stapled peptide 800ii on a mouse model of skin infection.

Peptide 800ii and mouse 800-CAP can reduce infection in mouse skin. Mice were infected with S. aureus SH1000 for 5 days in the presence of saline or 200 nM peptide. Infection was measured as viable bacteria in the skin and normalised to the saline control (100). The data are the means of 3 or 4 separate experiments, each performed in triplicate. Significance of difference from 100 was calculated using the one-sample t test. **p<0.001, *p<0.05.

FIG. 10 shows the circular dichroism spectra of peptides in PBS at pH7 with (A) 0%, (B) 10%, (C) 30% 2,2,2-trifluoroethanol. (D) % helical content vs % TFE for each peptide sequence.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, tissue culture, molecular biology, chemistry, biochemistry, recombinant DNA technology, and bioinformatics which are within the skill of the art. Such techniques are explained fully in the literature.

The terms “peptide” or “protein” are used interchangeably herein and refer to amino acids in a polymeric form of any length, linked together by peptide bonds.

The term “stability” may be defined as the stability of the peptide when incubated under appropriate conditions with one or more proteases; preferred proteases include, for example, trypsin, chymotrypsin or human serum, as described herein. In other words, stability may also be defined as resistance to proteases. The term “activity” may be defined as the ability of the peptide to inhibit bacterial adhesion.

The term “biofilm” shall be understood as a community of microorganisms formed within a matrix layer which are notoriously difficult to eradicate, and are often associated with microbial resistance due to the high cell density and exchange of genetic information.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

Peptide Design and Structure

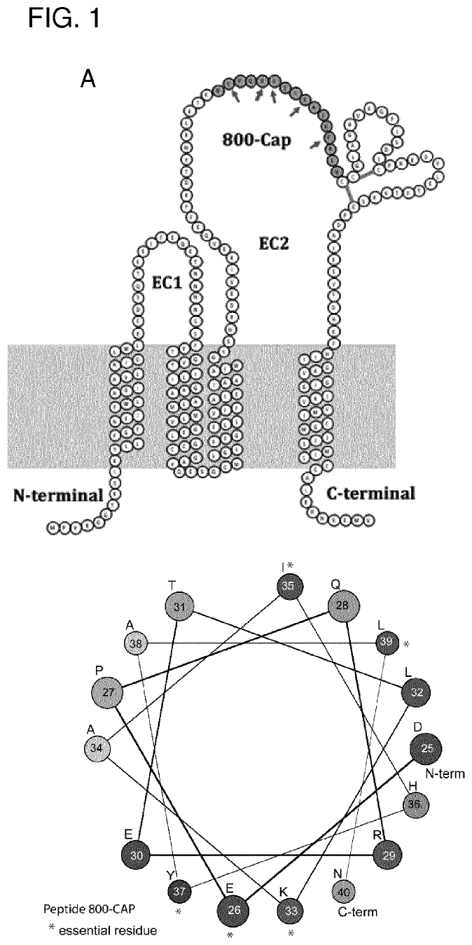

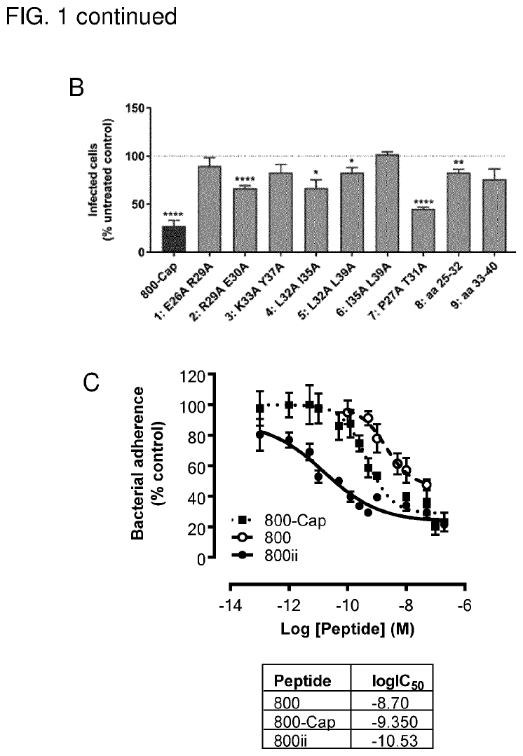

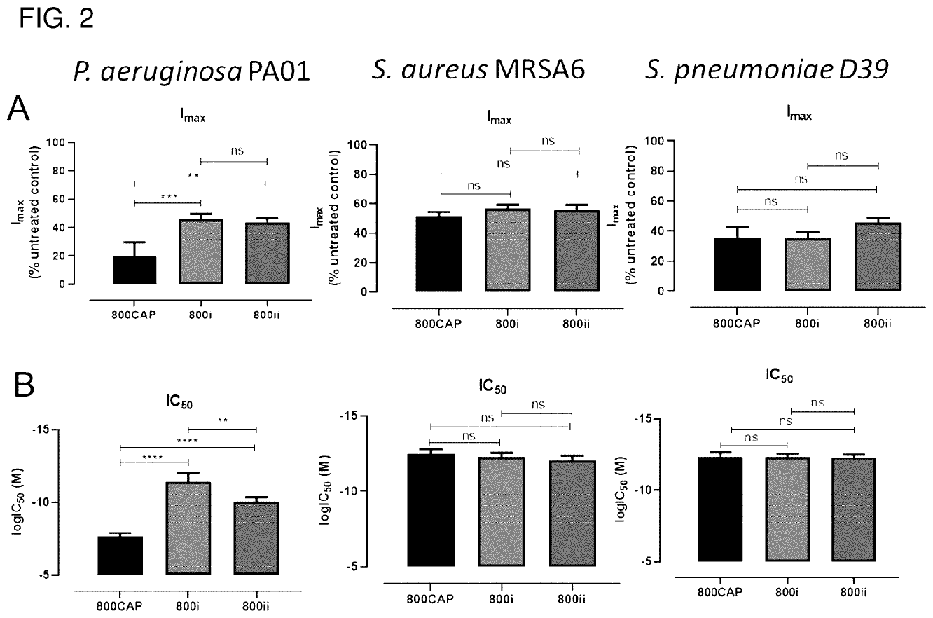

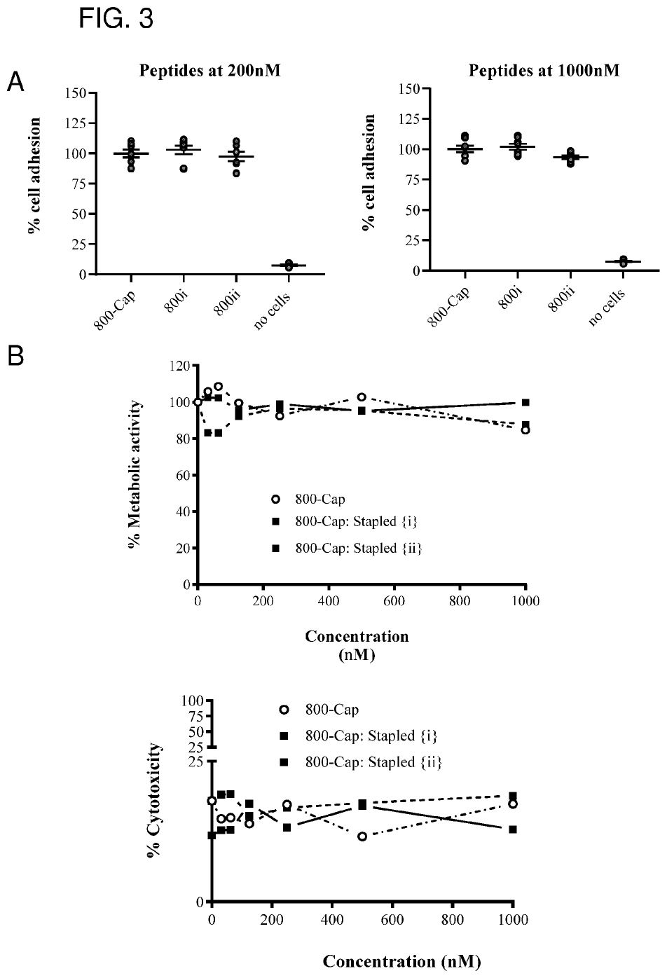

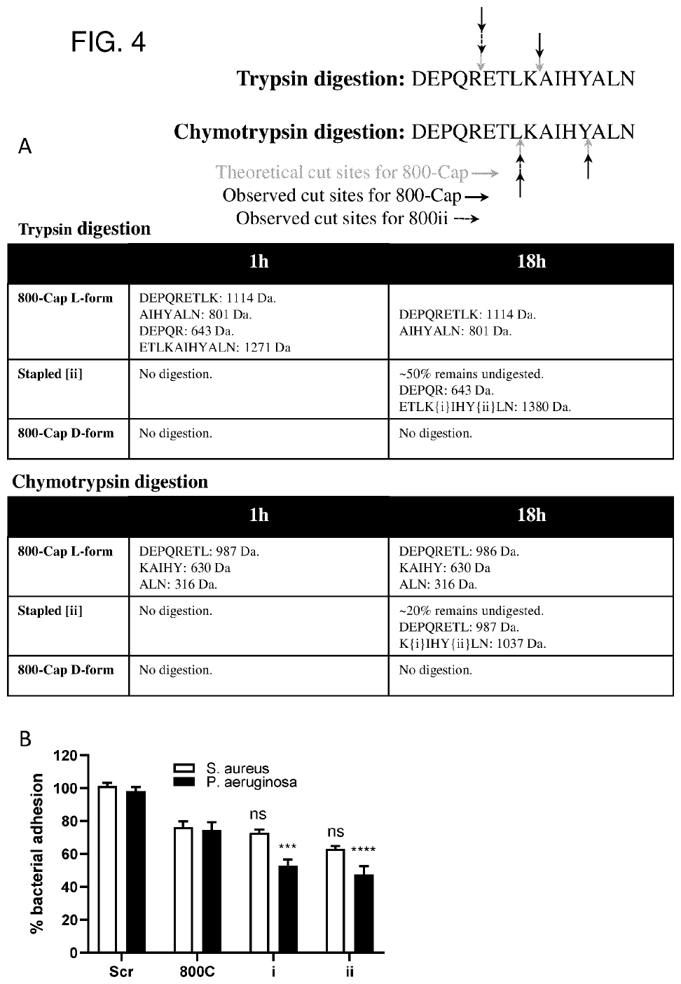

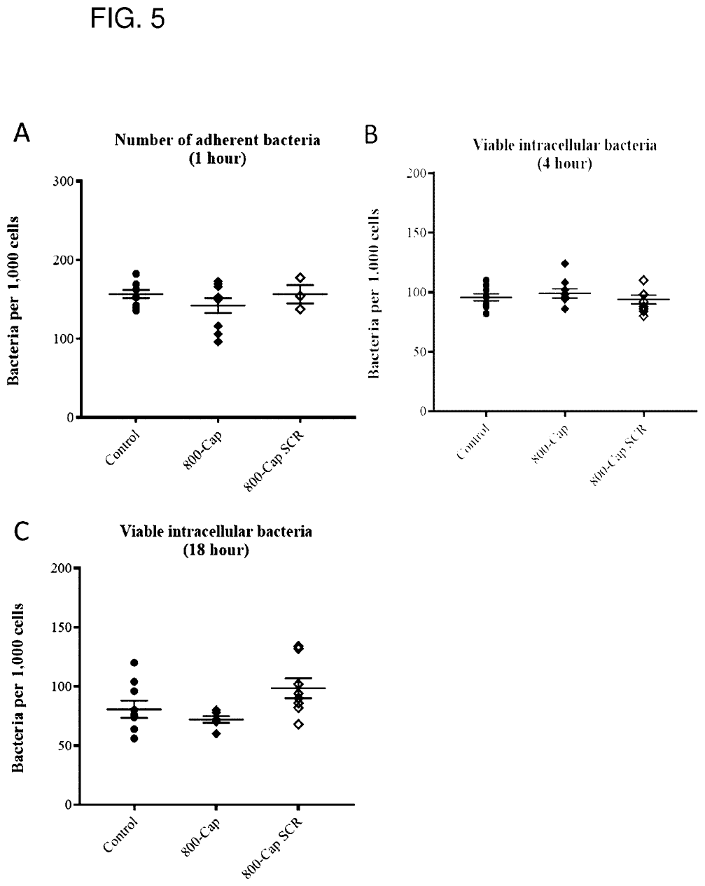

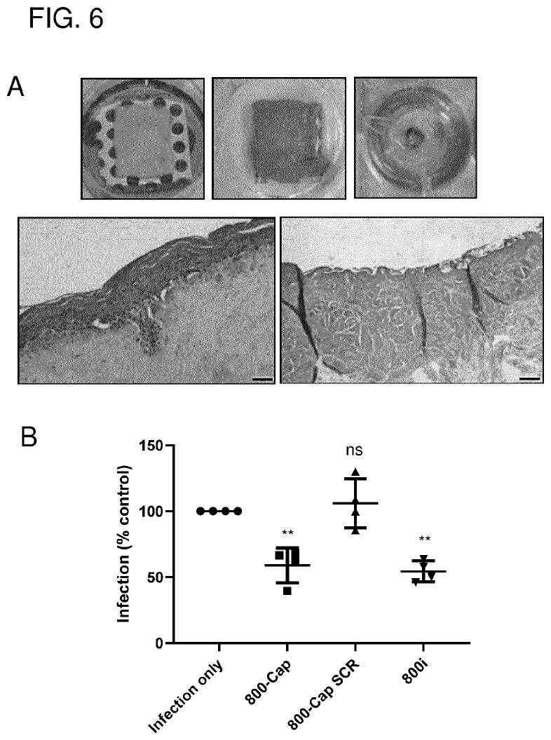

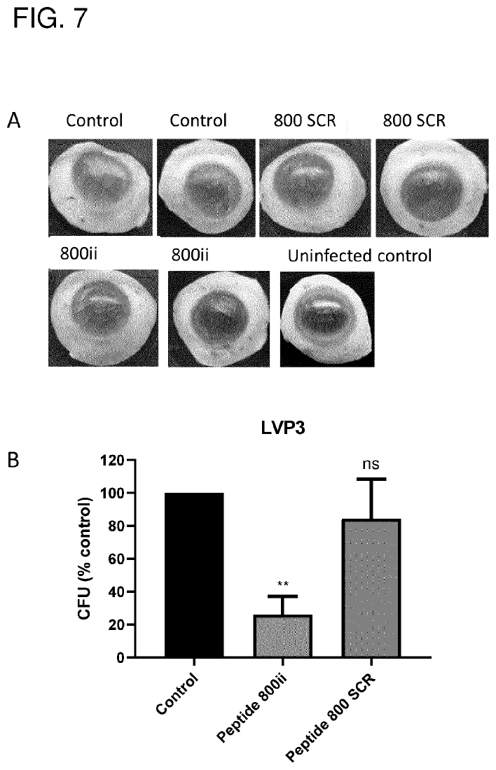

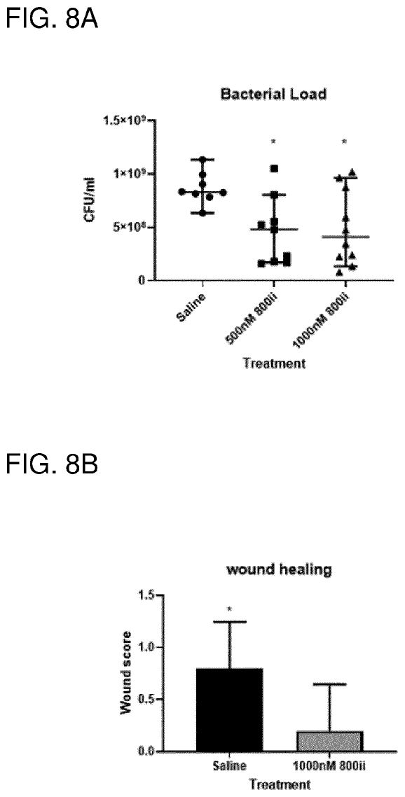

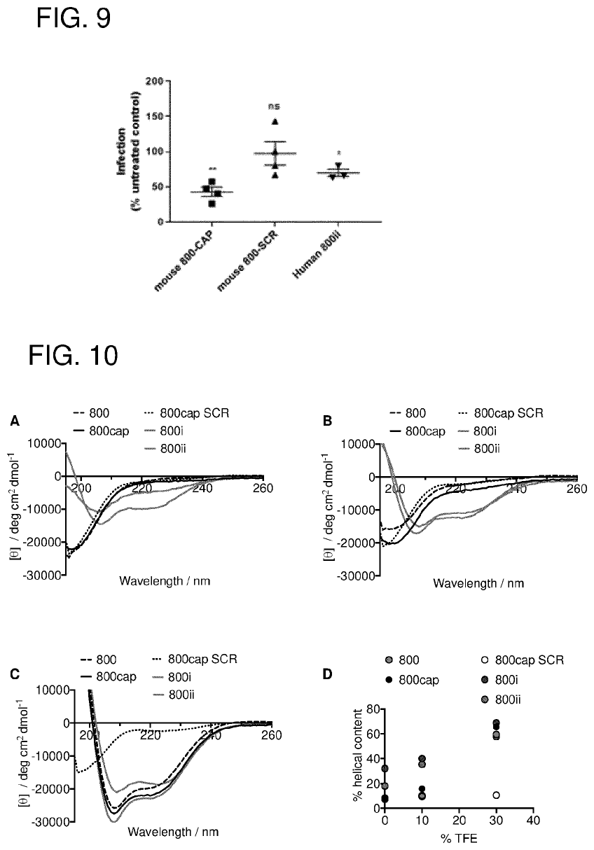

The region of CD9 EC2 from which peptide 800 (SEQ ID NO: 4) is derived (see FIG. 1A) is predicted to have an α-helical structure when modelled on the crystal structure of CD81. We predicted that a degree of helical structure would be important for the activity of peptide 800 by pre-organising the elements important for anti-adhesion activity. Therefore, we extended 800 with an N-terminal Asp residue (800-Cap, (SEQ ID NO: 5) Table 1) that should act to ‘cap’ the helix, helping to maintain helical structure in solution (10).

TABLE 1

| | Peptide sequences | | | | | | | | | | | | | | | |
|---|---|---|---|---|---|---|---|---|---|---|---|---|---|---|---|---|
| # | 1 | 2 | 3 | 4 | 5 | 6 | 7 | 8 | 9 | 10 | 11 | 12 | 13 | 14 | 15 | 16 |
| 800-Cap | D | E | P | Q | R | E | T | L | K | A | I | H | Y | A | L | N |
| (SEQ | | | | | | | | | | | | | | | | |
| ID NO: 5) | | | | | | | | | | | | | | | | |
| 800-Cap | Q | E | A | L | K | Y | N | R | A | E | T | P | L | D | I | H |
| SCR (SEQ | | | | | | | | | | | | | | | | |
| ID NO: 8) | | | | | | | | | | | | | | | | |
| Stapled | D | E | @ | Q | R | E | @ | L | K | A | I | H | Y | A | L | N |
| 800i (SEQ | | | | | | | | | | | | | | | | |
| ID NO: 2) | | | | | | | | | | | | | | | | |
| Stapled | D | E | P | Q | R | E | T | L | K | @ | I | H | Y | @ | L | N |
| 800ii (SEQ | | | | | | | | | | | | | | | | |
| ID NO: 3) | | | | | | | | | | | | | | | | |

@ could be = any amino acid capable of being stapled, which may include being modified to accommodate a staple (for example a (S)-2-(4-pentenyl)-alanine, a (S)-2-(7-octenyl)-alanine, or a (S)-2-(4-pentenyl)-glycine). In this particular case, @ = (S)-2-(4-pentenyl)-alanine.

A helical wheel representation of peptide 800-Cap (SEQ ID NO: 5) (FIG. 1A) shows the presumed location of residues Asp25-Asn40 of CD9 large extracellular domain (residues 1-16 of 800 peptide).

It will be understood that the following residues are equivalent:

Residue 137 of CD9—Residue 1 of 800 peptide (SEQ ID NO: 4)

Residue 138 of CD9—Residue 2 of 800 peptide

Residue 139 of CD9—Residue 3 of 800 peptide

Residue 140 of CD9—Residue 4 of 800 peptide

Residue 141 of CD9—Residue 5 of 800 peptide

Residue 142 of CD9—Residue 6 of 800 peptide

Residue 143 of CD9—Residue 7 of 800 peptide

Residue 144 of CD9—Residue 8 of 800 peptide

Residue 145 of CD9—Residue 9 of 800 peptide

Residue 146 of CD9—Residue 10 of 800 peptide

Residue 147 of CD9—Residue 11 of 800 peptide

Residue 148 of CD9—Residue 12 of 800 peptide

Residue 149 of CD9—Residue 13 of 800 peptide

Residue 150 of CD9—Residue 14 of 800 peptide

Residue 151 of CD9—Residue 15 of 800 peptide

Seven synthetic peptides were made with helix-preserving Ala residues replacing hydrophilic or hydrophobic residues at 1: Glu26/Arg29 (SEQ ID NO: 9), 2: Arg29/Glu30 (SEQ ID NO: 10), 3: Lys33/Tyr37 (SEQ ID NO: 12), 4: Leu32/Ile35 (SEQ ID NO: 13), 5: Leu32/Leu39 (SEQ ID NO: 14), 6: Ile35/Leu39 (SEQ ID NO: 15) and 7: Pro27/Thr31 (SEQ ID NO: 11).

These peptides were tested for inhibitory activity against S. aureus adhesion to human keratinocyte line HaCaT (FIG. 1B). Only peptide 7, Pro27/Thr31 (SEQ ID NO: 11), retained full activity at 200 nM, whereas peptides 1 and 6 (SEQ ID NO: 9 and 15) had no activity at this concentration as these peptides had mutations involved in intra-helical salt bonds or intra-helical hydrogen bonds. Shorter forms of peptide 800-Cap (SEQ ID NO: 17 and 18) appeared to have little or no inhibitory activity (FIG. 1B).

Both substituted residues in Pro27/Thr31 (SEQ ID NO: 11) were located on one face of the helical wheel (FIG. 1A), along with two Ala residues (Ala34 and Ala38). This face of the helix appeared to have no role in inhibitory activity and so residues here can be used for anchoring staples without compromising peptide function. These data suggest that the charged face of the 800 peptide is responsible for the anti-adhesion activity of the peptide and that helix stabilising mutations can be introduced into the hydrophobic face of the helix to enhance inhibitory properties. The addition of the capping Asp residue to peptide 800 (800-Cap (SEQ ID NO: 5), and then stapling between positions 10 and 14 (800ii, SEQ ID NO: 3) progressively increased the potency of the peptides (FIG. 1C) on S. aureus adherence to HaCaT cells.

Protease stability of the CD9 peptides was firstly assessed by digestion with trypsin, chymotrypsin or 25% human serum. The theoretical cut sites for the linear peptide 800-Cap (SEQ ID NO: 5) are shown (FIG. 4A). After 1 hour, 800-Cap (SEQ ID NO: 5) was completely digested by both proteases with at least one cleavage per peptide molecule (FIG. 4A). In contrast, 800ii (SEQ ID NO: 3) was not digested at all at 1 hour, and only partly digested at 18 hours with 50% and 20% still undigested in trypsin and chymotrypsin, respectively (FIG. 4A). Peptide 800-Cap synthesised with D-amino acids was not digested at either time point. Secondly, stability to bacterial cultures was tested by incubation with growing cultures of either S. aureus SH1000 or P. aeruginosa PA01. After 4 hrs incubation, bacteria were removed and the residual peptide activity tested by an infection assays using S. aureus SH1000 and HaCaT human keratinocytes (FIG. 4B). The activity of peptide 800-Cap (SEQ ID NO: 5) is partly degraded (˜50%) by both bacteria. In contrast, the stapled peptides 800i (SEQ ID NO: 2) and 800ii (SEQ ID NO: 3) are resistant to P. aeruginosa but not S. aureus. The stapling appears to protect against degradation by P. aeruginosa, perhaps by resistance to secreted proteases. However, S. aureus may secrete different proteases or may non-specifically bind the peptides.

Peptide Activity on a Human Corneal Cell Line

The activity of peptides 800-Cap (SEQ ID NO: 5), stapled 800i (SEQ ID NO: 2) and stapled 800ii (SEQ ID NO: 3) was tested against bacterial adhesion to human corneal HCE2 cells. The maximal level of inhibition (Iₘₐₓ) of adhesion was 40-50% for all peptides with S. aureus and S. pneumoniae, whereas 800-Cap (SEQ ID NO: 5) was significantly more effective against P. aeruginosa than the stapled peptides (FIG. 2A). The log IC₅₀ values for all peptides were in the same range (˜−12) against S. aureus and S. pneumoniae but higher for all peptides against P. aeruginosa (log IC₅₀−7.6-−11.4) (FIG. 2B). Again, 800-Cap (SEQ ID NO: 5) was significantly different to the stapled peptides (2-3 orders of magnitude less potent). This may reflect the resistance of the peptides to proteases secreted by P. aeruginosa, which would be expected to be higher for peptides protected by a staple, as observed for trypsin and chymotrypsin (FIG. 4B). The effects of peptides directly on the human HCE-2 cells was also tested. Peptides at up to 1000 nM had no effect on the attachment of HCE-2 cells to tissue culture plastic (FIG. 3A), had no cytotoxic activity and no effect on mitochondrial function (FIG. 3B).

Interactions with Phagocytic Cells

Phagocytosis is a defence mechanism against infecting microbes, in which they are opsonised or otherwise recognised by macrophages or neutrophils, engulfed and then usually destroyed. S. aureus SH1000 adherence to human monocyte-derived macrophages was shown not to be affected by 200 nM peptide 800-Cap (SEQ ID NO: 5) (FIG. 5A), suggesting that interactions with professional phagocytic cells may be different to interactions with epithelial cells. Bacteria were also internalised into the macrophages, but peptide treatment made no difference to the numbers of these at either 4 hours or 18 hours (FIG. 5B, C). It appears likely that professional phagocytes have different adherence and internalisation mechanisms when compared to epithelial cells and so CD9-derived peptides should not inhibit this element of the immune system.

Peptide Activity on a Complex Model of Human Skin Wound

We have previously published that peptide 800 (SEQ ID NO: 4) is active against S. aureus adhesion to a wound model of human skin (Ventress et al., 2016). FIG. 6A shows the skin growing in transwell inserts (top panel), with a wounded section of skin (top right). The skin forms an infection-resistant stratum corneum (FIG. 6A, lower left panel) that needs to be removed by burning to allow a productive infection (lower right panel). Peptide 800-Cap (SEQ ID NO: 5) and stapled peptide 800i (SEQ ID NO: 2) are also effective inhibitors of S. aureus SH1000 adhesion to wounded human skin (FIG. 6B). The scrambled control peptide for 800-Cap (SCR) (SEQ ID NO: 8) has no significant activity.

Peptide Activity on a Human Ex Vivo Corneal Infection Model

Human corneas can be infected with a variety of pathogens in an ex vivo infection model as in Pinnock et al., 2017 (8). We have tested stapled peptide 800ii (SEQ ID NO: 3) against a corneal clinical isolate of P. aeruginosa, LVP3. Infected corneas became increasingly opaque as the bacterial collagenases breakdown the clear layers of the corneum (FIG. 7A). 400 nM 800ii (SEQ ID NO: 3) but not control peptide 800-SCR (SEQ ID NO: 8) can significantly inhibit LVP3 adhesion to the cornea, decreasing opacity (FIG. 7A) and recoverable CU (FIG. 7B).

Peptide Activity on In Vivo Mouse Models

Peptide 800ii has been tested in in vivo models of microbial keratitis and skin infection to determine effects on bacterial infection and wound healing.

Peptide 800ii was tested in a mouse model of microbial keratitis, using a human clinical isolate of Pseudomonas aeruginosa obtained from a patient at LV Prasad Eye Institute, Hyderabad. A single application of either 500 nM or 1000 nM 800ii caused a significant reduction in the bacterial load after 24 hrs (FIG. 8A). In addition, 800ii at 1000 nM caused a significant enhancement of wound healing in uninfected corneas (FIG. 8B).

In a mouse model of skin infection, peptide 800ii or the mouse equivalent of 800-CAP at 200 nM was mixed with Staphylococcus aureus SH1000 and applied in a bandage to depilated mouse skin. After 5 days, the bacterial load was significantly reduced by both peptides relative to a scrambled peptide control (FIG. 9).

Helical Content and Helical Propensity of Peptides The helical content and the helical propensity of the peptides was measured in increasing concentrations of 2,2,2-trifluoroethanol (TFE), a solvent capable to stabilise helices (Luo and Baldwin, Biochemistry 1997, 36, 27, 8413-8421). Staple peptides 800i and 800ii are consistently more helical than all linear sequences in the absence and the presence of TFE. FIG. 10 shows the circular dichroism spectra of peptides in PBS pH7 with (A) 0%, (B) 10%, (C) 30% 2,2,2-trifluoroethanol. (D) % helical content vs % TFE for each peptide sequence.

Methods

Protease Stability

Protease stability was assessed using trypsin, chymotrypsin and human serum. Peptides at 100 μg/ml were incubated in 100 mM ammonium bicarbonate+2 mM calcium chloride were digested using trypsin (2 mg/ml), chymotrypsin (2 mg/ml) or human serum (25% (v/v)) for 1 or 18 hours. Digestion was performed at 37° C. for trypsin and human serum, and 25° C. for chymotrypsin. Reaction was stopped with 2.5% formic acid for trypsin and chymotrypsin. Human serum proteins were precipitated with acetonitrile. Supernatants were analysed on LC-MS. Stability to bacterial proteases was also analysed in cultures of S. aureus SH100 and P. aeruginosa PA01. 20 nM peptides in Keratinocyte-Serum Free Medium (KFSM) were incubated with mid-log phase cultures of bacteria for 4 hours. Bacteria were then removed by centrifugation and the supernatants used in bacterial adhesion assays, as described below.

Human Cell Line Culture

HCE-2 human corneal epithelial cells were cultured in flasks precoated with 0.01 mg/ml fibronectin, 0.03 mg/ml bovine collagen type I and 0.01 mg/mL bovine serum albumin (BSA), in KFSM supplemented with 0.05 mg/ml bovine pituitary extract (BPE), 5 ng/ml epidermal growth factor 500 ng/ml hydrocortisone and 0.005 mg/ml insulin. Subculturing was performed by trypsinisation and re-plating cells at a 1:3 dilution. For bacterial adhesion assays, HCE-2 cells (25, 000/well) were grown overnight in 96-well plates coated with only 0.03 mg/ml bovine collagen type I and 0.01 mg/mL bovine serum albumin, as S. aureus adheres strongly to fibronectin.

HaCaT human keratinocyte cells were cultured in DMEM supplemented with 10% (v/v) foetal bovine serum. Cells were subcultured by trypsinisation. For bacterial adhesion assays, HaCaT cells (10,000 cells/well) were grown overnight in 96-well plates.

Bacterial Growth Conditions

The S. aureus strains were laboratory strain SH1000 or local clinical isolates MRSA6 and S235. The P. aeruginosa strains used were laboratory strain PA01 and a clinical isolate from a corneal infection patient at LV Prasad Eye Institute, Hyderabad, India, LVP3. The S. pneumoniae used was laboratory strain, D39.

For infection assays, bacterial were cultured from a single agar plate colony overnight in 5 ml of the appropriate growth medium. Then cultures were harvested by centrifugation, washed with 10 ml PBS, re-suspended in growth medium and incubated to achieve an OD₆₀₀ equivalent to 5×10⁸ CFU/ml. Cultures were again harvested by centrifugation, washed twice with PBS and re-suspended in DMEM at an appropriate density.

Bacterial Adhesion Assays

Human cells in 96-well plates were washed with 200 ul HBSS and incubated with 200 μl 5% (w/v) BSA for 1 hour at 37° C., washed twice with 200 μl HBSS prior to incubation for 1 hour at 37° C. with peptides diluted in DMEM, or DMEM alone for control well. After removal of peptide solutions, wells were infected for 1 hour at 37° C. with 50 μl bacteria suspended in DMEM at optimised multiplicities of infection (MOI). After infection, wells were washed four times with 150 μl HBSS and finally re-suspended, with scraping, into 100 μl PBS. 1:10 dilutions were made and plated onto agar for colony-forming unit (CFU) counting.

Toxicity Assays

The possible effects of CD9 peptides on cellular adherence were measured by treating HCE-2 cells in suspension with 200 nM or 100 nM peptides. Briefly, HCE-2 cells were trypsinised for 7 minutes before trypsin was inactivated by the addition of KFSM. Cells were harvested by centrifugation, counted and re-suspended in fresh KFSM before being added to coated wells on a 96-well plate in the presence of 200 nM or 100 nM peptides or KFSM alone as control. After 1 hour at 37° C., wells were gently washed with 1 ml HBSS then cells were fixed with 4% paraformaldehyde for 20 minutes at room temperature. After washing twice with water, cells were stained with 0.1% (w/v) Crystal Violet in water for 30 minutes. Wells were then washed three times with water, air-dried and the dye solubilised with 2% SDS. The OD₅₇₀ was then measured.

Cytotoxicity was measured as intracellular lactate dehydrogenase (LDH) release into the supernatant. HCE-2 cells were plated in a coated 96-well plate at 25, 000 cells/well and grown overnight in KFSM in the presence of CD9 peptides at the indicated concentrations. The LDH assay was performed on supernatants using a Promega CytoTox 96 Non-radioactive cytotoxicity kit according to the manufacturer's instructions, and the OD₄₉₀ measured.

Metabolic activity was measured as NAD(P)H-dependent cellular oxidoreductase activity using the conversion of 3-(4,5-dimethylthiazol-2-yl)-2,5-diphenyltetrazolium bromide (MTT) to formazan. HCE-2 cells were plated in a coated 96-well plate at 25, 000 cells/well and grown overnight, and wells washed once with PBS. Peptides were added at the indicated concentrations in KFSM and then plates incubated overnight at 37° C. After removal of supernatants, 100 ul of 0.5 mg/ml MTT was added in 1:1 DMEM/F12 medium, and the plate incubated for 3 hours at 37° C. Wells were then washed with PBS and then 100 ul DMSO added to solubilised the formazan crystals. After 1 hour at room temperature, OD₅₄₀ was recorded.

Phagocytosis Assay

Peripheral blood mononuclear cells were isolated from the blood of healthy volunteers by density gradient centrifugation using Ficoll-Paque Plus (GE-Healthcare) as described previously (Dockrell et al., 2001) with ethics approval from the South Sheffield Research Ethics Committee (07/Q2305/7). MDM were resuspended at 10⁶ cells/ml in RPMI 1640 supplemented with 10% FBS for 24 hr at 37° C. in 5% CO2. After medium exchange, adherent monocytes were cultured for 8-11 days to differentiate into MDM as described previously by Dockrell et al., 2001 (9).

Skin Infection Model

The human 3D skin model was used as described in Ventress et al., 2016 (7).

Human Ex Vivo Corneal Infection Model

The human ex vivo corneal model was used as described in Pinnock et al., 2017(8).

Mouse In Vivo Microbial Keratitis Model

C57/Bl6 mice corneas were scored with a scalpel under anaesthetic and then treated with either 5 ul peptide 800ii (500 nM or 1000 nM) in sterile saline or saline alone for 5 min. P. aeruginosa clinical isolate LVP3 was added to both eyes in 5 ul saline. Bandages were applied and mice were left for 24 hrs in standard accommodation. Mice were sacrificed and eyes removed for histology (left eye) or counting of viable bacteria (right eye) after homogenisation. Bacterial loads are expressed as colony-forming units (CFU) per ml of homogenate. 10 mice were used for each condition.

Mouse In Vivo Corneal Wound Healing Model

To assess wound healing, C57/Bl6 mice corneas were scored with a scalpel under anaesthetic and then treated with either 5 ul peptide 800ii (1000 nM) in sterile saline or saline alone for 5 min. Bandages were applied and mice were left for 24 hrs in standard accommodation. Corneal permeability was assessed by adding fluorescein using a standard ophthalmological applicator strip and scored visually as either 0 (impermeable) or 1 (permeable).

Mouse In Vivo Skin Infection Model

C57/Bl6 mice were shaved to produce a bare area of approximately 2×2 cm. Depilatory cream was applied to depilate this area. A bandage was applied that contained a saline solution containing 200 nM of mouse 800-CAP, its scrambled control or human 800ii, and S. aureus SH1000. After 5 days, the mice were sacrificed and the depilated skin processed to release bacteria. The infection was assessed as CFU, and normalised to untreated control mice (=100). The experiment was performed 3 times using groups of 3 mice for each condition. Data are shown as the means of each separate experiment. Significance of the difference from 100 was calculated using a one-sample t test.

Circular Dichroism Measurements

Lyophilised peptides dissolved into PBS, pH7. Protein concentrations were quantified by UV absorption, using calculated extinction coefficients (ϵ₂₈₀), and the solutions adjusted to 20 μM final concentration with the same buffer, with or without the appropriate % (v/v) of 2,2,2-trifluoroethanol. Spectra were recorded in a 1 mm path length cell between 260 and 190 nm using a Chirascan Plus instrument (Applied Photophysics Ltd., UK) at 20° C. Spectra were baseline-corrected, smoothed (window size=5), and converted to units of mean residue ellipticity (MRE). The spectra were analysed to extract α-helix content for each protein, using:

$${\%}_{helix} = \frac{( {θ}_{222} - {θ}_{c} )}{( {θ}_{max} - {θ}_{c} )} \times 100$$

where θ₂₂₂ is the molar ellipticity of 222 nm; where θₘₐₓ is the expected θ₂₂₂ for a 100% helical peptide, θ_{c} is the expected θ₂₂₂ for a random coil, and:

θₘₐₓ=(−44,000+250T)(1−k/N)

θ_{c}=2220-53T

where N is the number of residues, k=2.4 and T=20° C.

### REFERENCES

1. Tong S Y C, Davis J S, Eichenberger E, Holland T L, Fowler V G Jr. Staphylococcus aureus Infections: Epidemiology, Pathophysiology, Clinical Manifestations, and Management. Clinical Microbiology Reviews. 2015; 28(3):603-61. doi: 10.1128/cmr.00134-14 PMID: WOS:000360495000004.
2. Zumaquero E, Munoz P, Cobo M, Lucena G, Pavon E J, Martin A, et al. Exosomes from human lymphoblastoid B cells express enzymatically active CD38 that is associated with signaling complexes containing CD81, Hsc-70 and Lyn. Experimental Cell Research. 2010; 316(16):2692-706. doi: 10.1016/j.yexcr.2010.05.032 PMID: WOS:000281305800016.
3. Monk P N, Partridge L J. Tetraspanins: gateways for infection. Infect Disord Drug Targets. 2012; 12 (1):4-17. PMID: 22034932
4. Hemler M E. Tetraspanin functions and associated microdomains. Nat Rev Mol Cell Biol. 2005; 6 (10):801-11. doi: 10.1038/nrm1736 PMID: 16314869.
5. Zhou G, Mo W J, Sebbel P, Min G W, Neubert T A, Glockshuber R, et al. Uroplakin Ia is the urothelial receptor for uropathogenic Escherichia coli: evidence from in vitro FimH binding. Journal of Cell Science. 2001; 114(22). PMID: WOS:000172594200015.
6. Hassuna N, Monk P N, Moseley G W, Partridge L J. Strategies for targeting tetraspanin proteins: potential therapeutic applications in microbial infections. BioDrugs. 2009; 23(6):341-59. doi: 10.2165/11315650-000000000-00000 PMID: 19894777
7. Ventress J K, Partridge L J, Read R C, Cozens D, MacNeil S, Monk P K. Peptides from Tetraspanin CD9 Are Potent Inhibitors of Staphylococcus Aureus Adherence to Keratinocytes. PLoS ONE. 2016: 11(7) e0160387. doi:10.1371/journal.pone.0160387.
8. Pinnock A, Shivshetty B N, Roy S, Rimmer S, Douglas I, MacNeil S, Garg P. Ex vivo rabbit and human corneas as models for bacterial and fungal keratitis. Graefe's Archive for Clinical and Experimental Ophthalmology 2017: 255: 333-342. doi: https://doi.org/10.1007/s00417-016-3546-0
9. Dockrell D H, Lee M, Lynch D H, Read R C. Immune-mediated phagocytosis and killing of Streptococcus pneumoniae are associated with direct and bystander macrophage apoptosis. J Infect Dis 2001: 184, 713-22. doi: 10.1086/323084
10. Forood B, Feliciano E J, Nambiar K P. Stabilization of α-helical structures in short peptides via end capping. Proc Natl Acad Sci 1993: 838-842. doi: 10.1073/pnas.90.3.838

1-24. (canceled)

25. A peptide comprising an amino acid sequence having at least 75% sequence identity to SEQ ID NO: 1, wherein the peptide comprises at least one staple between two or more residues equivalent to residues 3, 7, 10 or 14 of SEQ ID NO: 1.

26. The peptide according to claim 25, wherein the stability and/or the activity of the peptide is increased compared to a peptide consisting of any one of SEQ ID NO: 5, 6 or 7.

27. The peptide according to claim 25, wherein the peptide consists of an amino acid sequence defined by SEQ ID NO: 2 or SEQ ID NO: 3, or consists of an amino acid sequence consisting of SEQ ID NO:1, SEQ ID NO:2 or SEQ ID NO:3 having at least one substitution, deletion or addition.

28. The peptide according to claim 25, wherein the at least one staple comprises a covalent linkage between side chains of said two or more amino acids, preferably wherein at least one of the two or more amino acids is an amino acid having an unnatural side chain.

29. A peptide according to claim 25 for use in therapy.

30. The peptide according to claim 29, for treatment of a wound, preferably wherein the wound is a skin wound or a corneal wound.

31. The peptide according to claim 29, for use in reducing a bacterial load.

32. A peptide according to claim 25 for prevention and/or treatment of a microbial infection.

33. The peptide according to claim 32, wherein the microbial infection comprises Staphylococcus aureus, Streptococcus pneumoniae, Candida, Fusarium, Pseudomonas, Tricophyton, or Aspergillus.

34. The peptide according to claim 32, wherein prevention and/or clearance of a biofilm is increased when the peptide is used compared to when the peptide is not used.

35. A pharmaceutical composition comprising the peptide according to claim 25.

36. A method of treating a wound using the peptide according to claim 25 or the pharmaceutical composition comprising said peptide.

37. The method according to claim 36, wherein the wound is a skin wound or a corneal wound.

38. A method of treating a microbial infection using the peptide according to claim 25 or the pharmaceutical composition comprising said peptide.

39. The method according to claim 38, wherein the microbial infection comprises Staphylococcus aureus, Streptococcus pneumoniae, Candida, Fusarium, Pseudomonas, Tricophyton, or Aspergillus.

40. The method according to claim 36, wherein prevention and/or clearance of a biofilm is increased compared to when the peptide or the pharmaceutical composition is not used.

41. The method according to claim 36, further comprising use of at least one antimicrobial drug, preferably an antibiotic.

42. The method according to claim 36 wherein the peptide or pharmaceutical composition is administered topically, through inhalation, or by lavage fluid.


SEQUENCE LISTING (18 sequences)

SEQ ID NO: 1 - protein, 16 aa, Artificial Sequence
  Generic sequence
  VARIANT (3)..(3): Any amino acid
  VARIANT (7)..(7): Any amino acid
  VARIANT (10)..(10): Any amino acid
  VARIANT (14)..(14): Any amino acid
DEXQREXLKXIHYXLN

SEQ ID NO: 2 - protein, 16 aa, Artificial Sequence
  800i sequence
  VARIANT (3)..(3): (S)-2-(4-pentenyl) alanine
  VARIANT (7)..(7): (S)-2-(4-pentenyl) alanine
DEXQREXLKAIHYALN

SEQ ID NO: 3 - protein, 16 aa, Artificial Sequence
  800ii sequence
  VARIANT (10)..(10): (S)-2-(4-pentenyl) alanine
  VARIANT (14)..(14): (S)-2-(4-pentenyl) alanine
DEPQRETLKXIHYXLN

SEQ ID NO: 4 - protein, 15 aa, Artificial Sequence
  800 peptide sequence
EPQRETLKAIHYALN

SEQ ID NO: 5 - protein, 16 aa, Homo sapiens
DEPQRETLKAIHYALN

SEQ ID NO: 6 - protein, 16 aa, Mus musculus
DEPQRETLKAIHMALD

SEQ ID NO: 7 - protein, 16 aa, Sus scrofa
DDPQRETLKAIHYALD

SEQ ID NO: 8 - protein, 16 aa, Artificial Sequence
  800-Cap-SCR
QEALKYNRAETPLDIH

SEQ ID NO: 9 - protein, 16 aa, Artificial Sequence
  Glu26/Arg29 mutant 800-Cap peptide
DAPQAETLKAIHYALN

SEQ ID NO: 10 - protein, 16 aa, Artificial Sequence
  Arg29/Glu30 mutant 800-Cap peptide
DEPQAATLKAIHYALN

SEQ ID NO: 11 - protein, 16 aa, Artificial Sequence
  Pro27/Thr31 mutant 800-Cap peptide
DEAQREALKAIHYALN

SEQ ID NO: 12 - protein, 16 aa, Artificial Sequence
  Lys33/Tyr37 mutant 800-Cap peptide
DEPQRETLAAIHAALN

SEQ ID NO: 13 - protein, 16 aa, Artificial Sequence
  Leu32/Ile35 mutant 800-Cap peptide
DEPQRETAKAAHYALN

SEQ ID NO: 14 - protein, 16 aa, Artificial Sequence
  Leu32/Leu39 mutant 800-Cap peptide
DEPQRETAKAIHYAAN

SEQ ID NO: 15 - protein, 16 aa, Artificial Sequence
  Ile35/Leu39 mutant 800-Cap peptide
DEPQRETLKAAHYAAN

SEQ ID NO: 16 - protein, 81 aa, Homo sapiens
SHKDEVIKEVQEFYKDTYNKLKTKDEPQRETLKAIHYALNCCGLAGGVEQFISDICPKKDVLETFTVKSCPDAIKEVFDNK

SEQ ID NO: 17 - protein, 8 aa, Artificial Sequence
  first short form of 800-Cap
DEPQRETL

SEQ ID NO: 18 - protein, 8 aa, Artificial Sequence
  second short form of 800-Cap
KAIHYALN